(19) United States
(12) Patent Application Publication
(10) Pub. No.: US 2007/0086565 A1
(43) Pub. Date: Apr. 19, 2007

(54) Focally aligned CT detector

(76) Inventors: Richard A. Thompson, Clifton Park, NY (US); Jonathan D. Short, Saratoga Springs, NY (US); Abdelaziz Ikhlef, Waukesha, WI (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/163,298

(22) Filed: Oct. 13, 2005

(51) Int. Cl. H05G 1/60; A61B 6/00; G01N 23/00; G21K 1/12

(52) U.S. Cl. 378/19

(57) ABSTRACT

A focally aligned scintillator is constructed such that its scintillator walls are sloped so as to be angularly aligned with an x-ray source. The scintillator has a planar x-ray reception surface and a planar light emission surface, and a plurality of sidewalls connecting the planar x-ray reception surface and the planar light emission surface. The sidewalls extend non-perpendicularly between the planar x-ray reception surface and the planar light emission surface.

### BACKGROUND OF THE INVENTION

The present invention relates generally to diagnostic imaging and, more particularly, to a radiographic detector with focally aligned cells.

Typically, in computed tomography (CT) imaging systems, an x-ray source emits a fan-shaped beam toward a subject or object, such as a patient or a piece of luggage. Hereinafter, the terms “subject” and “object” shall include anything capable of being imaged. The beam, after being attenuated by the subject, impinges upon an array of radiation detectors. The intensity of the attenuated beam radiation received at the detector array is typically dependent upon the attenuation of the x-ray beam by the subject. Each detector element of the detector array produces a separate electrical signal indicative of the attenuated beam received by each detector element. The electrical signals are transmitted to a data processing system for analysis which ultimately produces an image.

Generally, the x-ray source and the detector array are rotated about the gantry within an imaging plane and around the subject. X-ray sources typically include x-ray tubes, which emit the x-ray beam at a focal point. X-ray detectors typically include a collimator for collimating x-ray beams received at the detector, a scintillator for converting x-rays to light energy adjacent the collimator, and photodiodes for receiving the light energy from the adjacent scintillator and producing electrical signals therefrom.

Typically, each scintillator of a scintillator array converts x-rays to light energy. Each scintillator discharges light energy to a photodiode adjacent thereto. Each photodiode detects the light energy and generates a corresponding electrical signal. The outputs of the photodiodes are then transmitted to the data processing system for image reconstruction.

Despite the numerous advancements achieved with known CT detectors, image quality remains a point of emphasis and an area in need of improvement. Specifically, there remains a need to improve image quality with a reduction in image artifacts. While image artifacts can be attributed to a number of factors, one issue faced with conventional CT detectors is the misalignment of the scintillators relative to the x-ray source, or to the post-patient collimator. The negative effects of a misaligned scintillator are illustrated in FIG. 8.

FIG. 8 is a cross-sectional view of a conventional CT detector 2. The detector includes a scintillator array 4 of scintillators 6. The scintillator array is placed atop a photodiode array (not shown) such that light emitted by the scintillator array in response to the reception of x-rays 7 is detected and processed by the photodiode array. For purposes of illustration, the scintillator array also includes a single misaligned scintillator 6(a). Conventional detector design also includes x-ray shielding elements 8. The shielding elements are designed to block x-rays and, as a result, typically block some x-rays 7(a) that do not pass through intercellular gaps 9, but fail to block x-rays 7(b) that do pass through the inter-cellular gaps 9.

The misalignment of the scintillator relative to the x-ray source causes x-rays to pass through different thicknesses of scintillator material and, as a result, causes spectral gain non-uniformities in the scintillator, for example, bone induced spectral artifacts. That is, for scintillators that are misaligned relative to others, the path-lengths of x-rays will be different from other scintillators. This causes those misaligned scintillators to have a different response with respect to spectrum than the properly aligned scintillators.

In other words, x-rays that pass through the inter-scintillator gaps have a different path-length than those that pass through the scintillator alone. This difference in path-lengths causes the misaligned scintillator to have a different response relative to the neighboring and properly aligned scintillators. Moreover, misaligned shield elements can also contribute to the spectral non-linearity that occurs when the scintillators are misaligned. As a result, conventional CT detectors are susceptible to detector cell misalignment-induced artifacts, such as rings, bands and center artifacts.

Therefore, it would be desirable to design a CT detector that is less prone to misalignment-induced artifacts. It would be further desirable to have a CT detector having detector cells that are consistently aligned relative to the x-ray source of a radiographic imaging system.

### BRIEF DESCRIPTION OF THE INVENTION

The present invention is a directed a focally aligned CT detector that overcomes the aforementioned drawbacks. The CT detector is constructed such that scintillator walls are sloped so as to be angularly aligned with an x-ray source. In this regard, the CT detector is less prone to spectral artifacts associated with detector cell misalignment.

Therefore, in accordance with one aspect, the present invention includes a scintillator having a planar x-ray reception surface and a planar light emission surface. The scintillator also has a plurality of sidewalls connecting the planar x-ray reception surface and the planar light emission surface. The sidewalls extend non-perpendicularly between the planar x-ray reception surface and the planar light emission surface.

In accordance with another aspect of the invention, a radiographic detector includes a photodiode array including a plurality of photodiodes configured to output electrical signals in response to sensed light. Each photodiode has a planar light detection surface. The detector further has a scintillator array including a plurality of scintillators configured to emit light in response to the reception of x-rays. Each scintillator has sidewalls that are askew relative to the planar light detection surface of a respective photodiode.

According to another aspect, the present invention includes a CT system having a rotatable gantry. The gantry has an opening to receive an object to be scanned. The system also has an x-ray source configured to project an x-ray fan beam toward the object at a given projection angle and a scintillator array having a plurality of scintillator cells configured to convert x-ray energy to light. Each scintillator each cell is defined by off-centered sidewalls that extend along an angle that is parallel to the given projection angle. A photodiode array is optically coupled to the scintillator array and includes a plurality of photodiodes configured to detect light emitted from the scintillator array and provide an electrical signal output. The system further has a data acquisition system (DAS) connected to the photodiode array and configured to receive the electrical signal output of the photodiode array, and an image reconstructor connected to the DAS and configured to reconstruct an image of the object from the photodiode array electrical signal output received by the DAS.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a pictorial view of a CT imaging system.

FIG. 2 is a block schematic diagram of the system illustrated in FIG. 1.

FIG. 3 is a perspective view of one embodiment of a CT system detector array.

FIG. 4 is a perspective view of one embodiment of a detector.

FIG. 5 is illustrative of various configurations of the detector in FIG. 4 in a four-slice mode.

FIG. 6 is a partial cross-sectional view of a CT detector according to the present invention.

FIG. 7 is a pictorial view of a CT system for use with a non-invasive package inspection system.

FIG. 8 is a cross-sectional view of a conventional CT detector.

### DETAILED DESCRIPTION OF THE PREFERRED EMBODIMENT

The operating environment of the present invention is described with respect to a four-slice computed tomography (CT) system. However, it will be appreciated by those skilled in the art that the present invention is equally applicable for use with single-slice or other multi-slice configurations. Moreover, the present invention will be described with respect to the detection and conversion of x-rays. However, one skilled in the art will further appreciate that the present invention is equally applicable for the detection and conversion of other high frequency electromagnetic energy. The present invention will be described with respect to a “third generation” CT scanner, but is equally applicable with other CT systems. Also, the present invention is also believed to be applicable to detectors of other radiographic imaging systems, such as x-ray scanners.

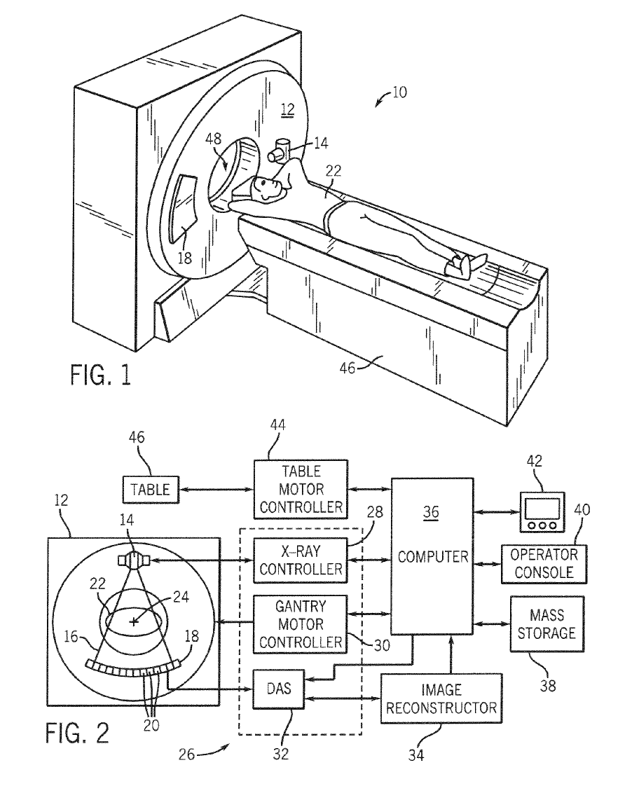

Referring to FIGS. 1 and 2, a computed tomography (CT) imaging system 10 is shown as including a gantry 12 representative of a “third generation” CT scanner. Gantry 12 has an x-ray source 14 that projects a beam of x-rays 16 toward a detector array 18 on the opposite side of the gantry 12. Detector array 18 is formed by a plurality of detectors 20 which together sense the projected x-rays that pass through a medical patient 22. Each detector 20 produces an electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. A data acquisition system (DAS) 32 in control mechanism 26 samples analog data from detectors 20 and converts the data to digital signals for subsequent processing. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves portions of patient 22 through a gantry opening 48.

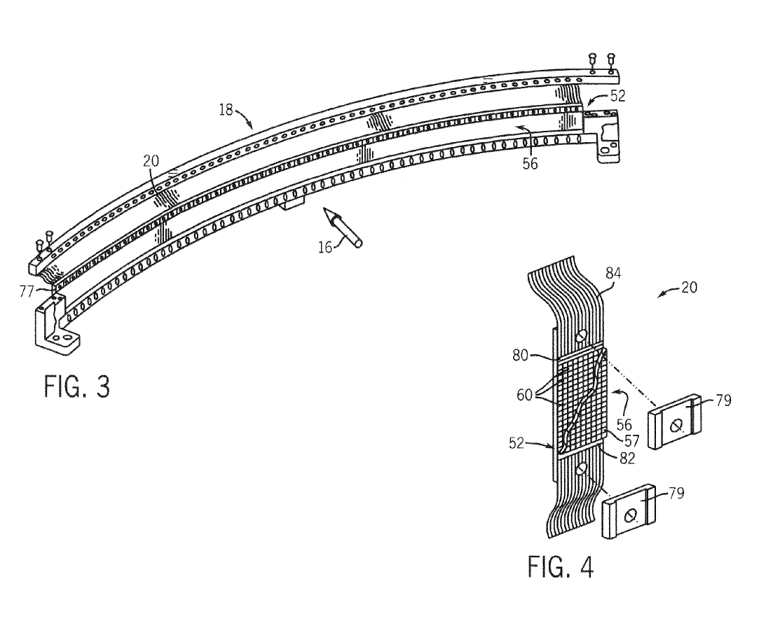

As shown in FIGS. 3 and 4, detector array 18 includes a plurality of scintillators 57 forming a scintillator array 56. A post-patient collimator (not shown) is positioned above scintillator array 56 to collimate x-ray beams 16 before such beams impinge upon scintillator array 56.

In one embodiment, shown in FIG. 3, detector array 18 includes 57 detectors 20, each detector 20 having an array size of 16×16. As a result, array 18 has 16 rows and 912 columns (16×57 detectors) which allows 16 simultaneous slices of data to be collected with each rotation of gantry 12.

Switch arrays 80 and 82, FIG. 4, are multi-dimensional semiconductor arrays coupled between scintillator array 56 and DAS 32. Switch arrays 80 and 82 include a plurality of field effect transistors (FET) (not shown) arranged as multi-dimensional array. The FET array includes a number of electrical leads connected to each of the respective photodiodes 60 and a number of output leads electrically connected to DAS 32 via a flexible electrical interface 84. Particularly, about one-half of photodiode outputs are electrically connected to switch 80 with the other one-half of photodiode outputs electrically connected to switch 82. Additionally, a reflector layer (not shown) may be interposed between each scintillator 57 to reduce light scattering from adjacent scintillators. Each detector 20 is secured to a detector frame 77, FIG. 3, by mounting brackets 79.

Switch arrays 80 and 82 further include a decoder (not shown) that enables, disables, or combines photodiode outputs in accordance with a desired number of slices and slice resolutions for each slice. Decoder, in one embodiment, is a decoder chip or a FET controller as known in the art. Decoder includes a plurality of output and control lines coupled to switch arrays 80 and 82 and DAS 32. In one embodiment defined as a 16 slice mode, decoder enables switch arrays 80 and 82 so that all rows of the photodiode array 52 are activated, resulting in 16 simultaneous slices of data for processing by DAS 32. Of course, many other slice combinations are possible. For example, decoder may also select from other slice modes, including one, two, and four-slice modes.

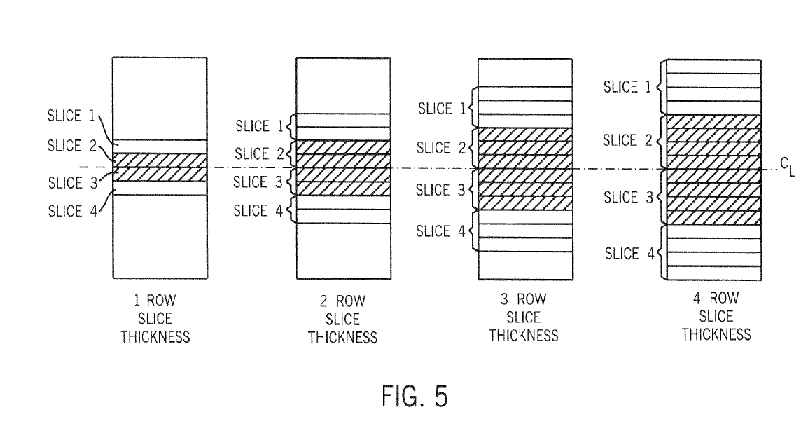

As shown in FIG. 5, by transmitting the appropriate decoder instructions, switch arrays 80 and 82 can be configured in the four-slice mode so that the data is collected from four slices of one or more rows of photodiode array 52. Depending upon the specific configuration of switch arrays 80 and 82, various combinations of photodiodes 60 can be enabled, disabled, or combined so that the slice thickness may consist of one, two, three, or four rows of scintillator array elements 57. Additional examples include, a single slice mode including one slice with slices ranging from 1.25 mm thick to 20 mm thick, and a two slice mode including two slices with slices ranging from 1.25 mm thick to 10 mm thick. Additional modes beyond those described are contemplated.

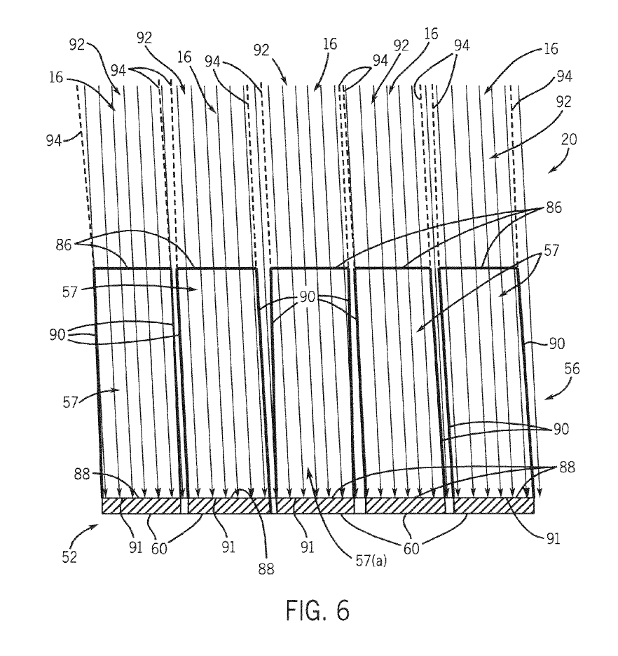

Referring now to FIG. 6, a cross-sectional view of a CT detector 20 according to the present invention is illustrated. For purposes of illustration, only five scintillators and photodiodes are shown, but one skilled in the art will appreciate that a CT detector may include several more such scintillators and photodiodes. Moreover, as is known, the scintillator and photodiode arrays are 2D arrays. As illustrated and described above, detector 20 includes a scintillator array 56 comprised of a plurality of scintillators 57 that illuminate upon the reception of x-ray energy. That illumination is detected by photodiodes 60 of photodiode array 52. In this regard, each scintillator 57 has a planar x-ray reception surface 86 and a planar light emission surface 88. Surfaces 86, 88 are connected to one another by scintillator septa or sidewalls 90. As shown, the sidewalls 90 are angled relative to the x-ray reception and light emission surfaces. As a result of the angularity of the sidewalls, the x-ray reception surface 86 of a scintillator 57 is offset from the light emission surface 88 of the scintillator 57.

The angled sidewalls 90 are angled so that the scintillators are focused on the x-ray source (not shown). In this regard, the sidewalls are sloped parallel to the x-ray paths 16. This sloping results in the sidewalls being oriented non-perpendicularly relative to the x-ray reception and light emission surfaces. Moreover, the septa are angled relative to the faces 91 of the photodiodes 52. As a result, x-ray path is relatively uniform and constant between scintillators of the scintillator array. This is particularly advantageous for a misaligned scintillator such as that illustrated by scintillator 57(a). In other words, the spectral response is less sensitive to scintillator misalignment because of less variance in path-length for a misaligned scintillator.

Still referring to FIG. 6, CT detector 20 preferably includes a collimator 92 that is collectively formed by an array of collimator elements or plates 94. Preferably, each collimator plate is arranged as an extension of a respective scintillator sidewall. Thus, similar to the scintillator array, the collimator grid is also aligned with the x-ray source. Additionally, it is contemplated that detector 20 may be constructed to have shielding elements (not shown) to provide additional x-ray collimation and isolation. Moreover, it is preferred that scintillator gaps 90 be filled with a light reflective epoxy or other material to reduce optical cross-talk between scintillators. The collimator plates 94 collectively form a 1D collimator 92.

It is contemplated that the scintillator construction described above can be achieved according to one of a number of fabrication techniques, or combinations thereof. In this regard, the scintillator may be formed through casting of scintillator material. Alternately, conventional molding techniques may be used. Additionally, it is contemplated that mechanical or chemical cutting techniques may be used. Moreover, it is contemplated that electromagnetic ablation, such as with a laser, may also be used. Regardless of fabrication technique, the scintillator walls are constructed so as to slope toward an x-ray source of a radiographic imaging system when situated in a detector assembly. Advantageously, this results in the sidewalls themselves not being exposed to primary radiation during data acquisition.

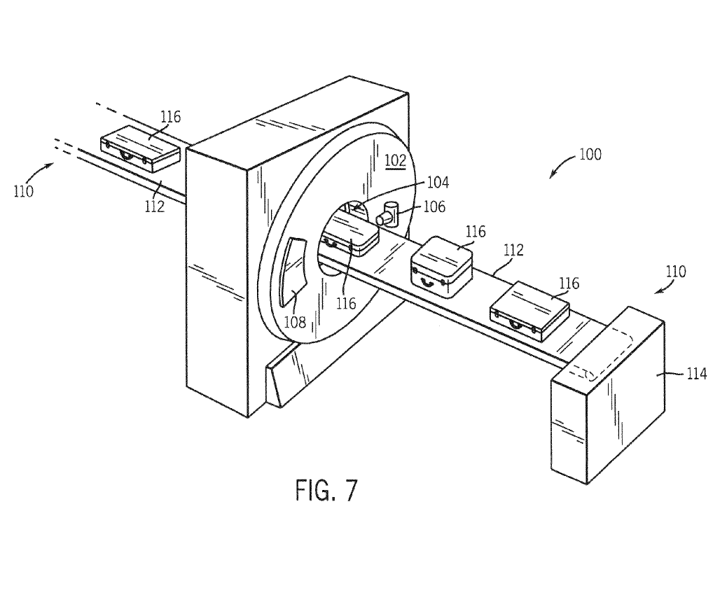

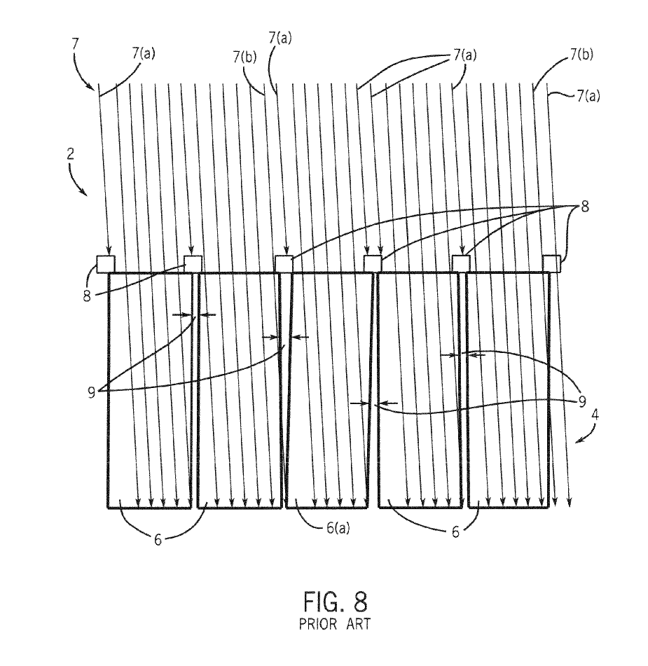

Referring now to FIG. 7, parcel/package/baggage inspection system 100 includes a rotatable gantry 102 having an opening 104 therein through which packages, parcels or pieces of baggage may pass. The rotatable gantry 102 houses a high frequency electromagnetic energy source 106 as well as a detector assembly 108 having scintillator arrays comprised of scintillator cells similar to that described above. A conveyor system 110 is also provided and includes a conveyor belt 112 supported by structure 114 to automatically and continuously pass packages or baggage pieces 116 through opening 104 to be scanned. Objects 116 are fed through opening 104 by conveyor belt 112, imaging data is then acquired, and the conveyor belt 112 removes the packages 116 from opening 104 in a controlled and continuous manner. As a result, postal inspectors, baggage handlers, and other security personnel may non-invasively inspect the contents of packages 116 for explosives, knives, guns, contraband, etc.

Therefore, in accordance with one aspect, the present invention includes a scintillator having a planar x-ray reception surface and a planar light emission surface. The scintillator also has a plurality of sidewalls connecting the planar x-ray reception surface and the planar light emission surface. The sidewalls extend non-perpendicularly between the planar x-ray reception surface and the planar light emission surface.

In accordance with another aspect of the invention, a radiographic detector includes a photodiode array including a plurality of photodiodes configured to output electrical signals in response to sensed light. Each photodiode has a planar light detection surface. The detector further has a scintillator array including a plurality of scintillators configured to emit light in response to the reception of x-rays. Each scintillator has sidewalls that are askew relative to the planar light detection surface of a respective photodiode.

According to another aspect, the present invention includes a CT system having a rotatable gantry. The gantry has an opening to receive an object to be scanned. The system also has an x-ray source configured to project an x-ray fan beam toward the object at a given projection angle and a scintillator array having a plurality of scintillator cells configured to convert x-ray energy to light. Each scintillator each cell is defined by off-centered sidewalls that extend along an angle that is parallel to the given projection angle. A photodiode array is optically coupled to the scintillator array and includes a plurality of photodiodes configured to detect light emitted from the scintillator array and provide an electrical signal output. The system further has a data acquisition system (DAS) connected to the photodiode array and configured to receive the electrical signal output of the photodiode array, and an image reconstructor connected to the DAS and configured to reconstruct an image of the object from the photodiode array electrical signal output received by the DAS.

The present invention has been described in terms of the preferred embodiment, and it is recognized that equivalents, alternatives, and modifications, aside from those expressly stated, are possible and within the scope of the appending claims.

What is claimed is:

1. A scintillator comprising:
a planar x-ray reception surface and a planar light emission surface; and
a plurality of sidewalls connecting the planar x-ray reception surface and the planar light emission surface, the sidewalls extending non-perpendicularly between the planar x-ray reception surface and the planar light emission surface.

2. The scintillator of claim 1 wherein the sidewalls are angularly positioned between the planar x-ray reception surface and the planar light emission surface so that the sidewalls are aligned with an x-ray source during radiographic imaging.

3. The scintillator of claim 2 wherein the planar x-ray reception surface is linearly offset from the planar light emission surface.

4. The scintillator of claim 1 formed by casting scintillator material.

5. The scintillator of claim 1 formed by molding scintillator material.

6. The scintillator of claim 1 formed by cutting of a scintillator bulk.

7. The scintillator of claim 1 formed by electromagnetic ablation with a laser.

8. The scintillator of claim 1 incorporated into a detector assembly of a CT scanner.

9. A radiographic detector comprising:
a photodiode array including a plurality of photodiodes configured to output electrical signals in response to sensed light, each photodiode having a planar light detection surface; and
a scintillator array including a plurality of scintillators configured to emit light in response to reception of x-rays, each scintillator having sidewalls that are askew relative to the planar light detection surface of a respective photodiode.

10. The radiographic detector of claim 9 wherein the sidewalls of each scintillator are aligned with an x-ray source designed to emit a fan beam of x-rays during radiographic imaging.

11. The radiographic detector of claim 10 further comprising a collimator grid having collimator plates aligned in parallel with the sidewalls of the scintillators.

12. The radiographic detector of claim 10 wherein the sidewalls of each scintillator connect an x-ray reception surface to a light emission surface, and wherein the x-ray reception surface is linearly offset from the light emission surface.

13. The radiographic detector of claim 12 wherein the x-ray reception surface of a scintillator has a surface area equal to that of the light emission surface of the scintillator.

14. The radiographic detector of claim 9 incorporated in a CT scanner.

15. A computed tomography (CT) system comprising:
a gantry having an opening defined therein to receive an object to be scanned;
an x-ray source configured to project an x-ray fan beam toward the object to be scanned at a given projection angle;
a scintillator array having a plurality of scintillator cells configured to convert x-ray energy to light, each cell defined by off-centered sidewalls that extend along an angle that is parallel to the given projection angle;
a photodiode array optically coupled to the scintillator array and comprising a plurality of photodiodes configured to detect light emitted from the scintillator array and provide an electrical signal output;
a data acquisition system (DAS) connected to the photodiode array and configured to receive the electrical signal output of the photodiode array; and
an image reconstructor connected to the DAS and configured to reconstruct an image of the object from the photodiode array electrical signal output received by the DAS.

16. The CT system of claim 15 further comprising a collimator grid including collimator plates that are aligned with the off-centered sidewalls of the scintillator cells.

17. The CT system of claim 15 wherein the scintillator array is formed by casting scintillator material.

18. The CT system of claim 15 wherein the scintillator array is formed by molding scintillator material.

19. The CT system of claim 15 wherein the scintillator array is formed by cutting of a scintillator bulk.

20. The CT system of claim 15 configured to acquire CT data of a medical patient.

21. The CT system of claim 15 configured to acquire CT data of at least one of a package, a parcel, and a piece of luggage.

22. The CT system of claim 15 wherein the gantry is a rotatable gantry.
